(19) United States
(12) Patent Application Publication
(10) Pub. No.: US 2012/0237607 A1
(43) Pub. Date: Sep. 20, 2012

(54) Immortalized cell compositions and compositions derived therefrom

(76) Inventors: Randall G. Rupp, Swanton, VT (US); Linda O. Palladino, Stormville, NY (US)

(21) Appl. No.: 13/407,804

(22) Filed: Feb. 29, 2012

Related U.S. Application Data
(60) Provisional application No. 61/465,098, filed on Mar. 14, 2011.

(51) Int. Cl. A61K 35/12; C12N 5/0735

(52) U.S. Cl. 424/520; 435/371

(57) ABSTRACT

The invention is directed to immortalized cell compositions and compositions derived therefrom. The invention is further directed to methods of making and using such immortalized cell compositions and compositions derived therefrom. Such immortalized cell compositions include but are not limited to Immortalized Amnion-derived Multipotent Progenitor cells (herein referred to as I-AMP cells) and conditioned media derived therefrom (herein referred to as I-Amnion-derived Cellular Cytokine Solution or I-ACCS).

### CROSS-REFERENCE TO RELATED APPLICATIONS

This application claims priority under 35 USC §119(e) of U.S. Provisional Application No. 61/465,098, filed Mar. 14, 2011, the entirety of which is incorporated herein by reference.

### FIELD OF THE INVENTION

The field of the invention is directed to immortalized cell compositions. The field of the invention is further directed to methods of making such immortalized cell compositions and methods of using such immortalized cell compositions. Such immortalized cell compositions include but are not limited to Immortalized Amnion-derived Multipotent Progenitor cells (herein referred to as I-AMP cells) and conditioned media derived therefrom (herein referred to as I-Amnion-derived Cellular Cytokine Solution or I-ACCS). The field of the invention is further directed to methods of making and using I-AMP cells and I-ACCS.

### DESCRIPTION OF RELATED ART

Lei, K-J., et al, Molecular Endocrinology 6:703-712, 1992) describe the immortalization of virus-free human cytotrophoblastic placental cell lines that express tissue-specific function.

Zhang, X., et al, (Biochem Biophys Res Commun, 351(4):853-9, 2006) describe the successful immortalization of mesenchymal progenitor cells derived from human placenta and the differentiation abilities of immortalized cells.

Wang, Y-L., et al (Molecular Human Reproduction, 12(7):451-60, 2006) describe immortalization of normal human cytotrophoblast cells by reconstitution of telomeric reverse transcriptase activity.

Feng, J. Y., et al., (Cancer Genet Cytogenet, 163(1):30-7, 2005) describe immortalization of human extravillous cytotrophoblasts by human papilloma virus gene E6E7: sequential cytogenetic and molecular genetic characterization.

He, S., et al (Annu Rev Cell Div Biol 2009, 25:377-406) and Orford, K. and Scadden, D. (Nature Rev, 2008, 9:115-128) both review the mechanisms of stem cell self-renewal and genetic insights into cell cycle.

### BACKGROUND OF THE INVENTION

A unique population of multipotent cells, termed Amnion-derived Multipotent Progenitor cells (AMP cells) have been selected from amnion epithelial cell populations (see U.S. Publication Nos. 2006-0222634 and 2007-0231297, each of which is incorporated by reference herein). AMP cells have not been cultured in the presence of any non-human animal-derived substances or products, making them and cell products derived from them suitable for human clinical use as they are not xeno-contaminated. This novel population of cells has the characteristic of secreting a unique combination of physiologically relevant cytokines in a physiologically relevant temporal manner and at physiological levels into the extracellular space or into surrounding culture media. In addition, AMP cells grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the AMP cells react with antibodies to low molecular weight cytokeratins, confirming their epithelial nature. Freshly isolated amnion epithelial cells, from which AMP cells are selected, will not react with antibodies to the stem/progenitor cell markers c-kit (CD117) and Thy-1 (CD90). Selected AMP cells remain c-kit negative, while expression of CD90 increases.

Another characteristic of AMP cells is that they are not immortal, meaning that they can only undergo between 6-12 population doublings before they die. While this characteristic may be desirable from a cell transplantation perspective, there are circumstances in which it would be preferable to have an AMP cell population which is immortal, particularly in the context of manufacturing AMP cell-derived products, yet maintain the AMP cell population's desirable characteristics. The instant invention provides for such novel immortalized cell populations, methods of making them, as well as their uses.

### BRIEF SUMMARY OF THE INVENTION

It is an object of the present invention to provide a composition comprising a population of AMP cells which comprises immortalized AMP cells. Such cells, termed Immortalized AMP cells (I-AMP cells) possess most of the desirable features and characteristics of AMP cells except that they are able to undergo greater than 12 population doublings, preferably greater than 20 population doublings, and most preferably greater than 50 population doublings. One important characteristic of AMP cells is that they are not transformed, meaning they have are not tumorigenic. It is desirable to maintain this characteristic in I-AMP cells.

AMP cells are useful in the manufacture of cell-derived products, or are products themselves. One such cell-derived product, termed “Amnion-derived Cellular Cytokine Solution” (ACCS), is derived from AMP cells (for details, see U.S. Pat. No. 8,088,732 and No. 8,058,066, both incorporated by reference herein). However, because AMP cells are not immortal, it is necessary to continually replace them in order to derive products from them. This requires obtaining placentas, stripping the amnion from the placenta, recovering the amnion epithelial cells lining the amnion and selecting AMP cells from the amnion epithelial cells. This process is expensive and time consuming and potentially limited by the availability of placentas to support the large scale manufacture of ACCS and other AMP cell-derived products. Because I-AMP cells are capable of population doublings significantly greater than those obtainable with AMP cells (which are typically capable of only 6-12 population doublings), there is significantly less need to repeatedly obtain placentas for processing and selection of AMP cells to replace those that have exhausted their population doubling capacity. The creation of I-AMP cells also eliminates the inherent variability that exists between different placentas and the AMP cells obtained therefrom. In fact, for the first time, it becomes possible to create an I-AMP cell line that can be banked, optionally under Good Manufacturing Procedure (GMP) conditions, thus providing a consistent and continual source of I-AMP cells for seeding cell culture manufacturing processes useful for generating clinical grade product. Because the cells are banked from a single population (optionally a clonal population) of cells, the need for repeated testing and characterization each time new AMP cells are selected from a new placenta is significantly reduced and possibly eliminated, thus allowing for simpler manufacturing processes at significantly lower cost.

Accordingly, a first aspect of the invention is a composition comprising a population of Immortalized Amnion-derived Multipotent Progenitor (I-AMP) cells, wherein the I-AMP cells are capable of greater than 12 population doublings.

A second aspect of the invention is a method of making I-AMP cells comprising: a) isolating amnion epithelial cells from the amnion of a placenta; b) selecting AMP cells from the amnion epithelial cells; and c) manipulating the AMP cells to immortalize them, wherein immortalization is characterized by the I-AMP cells being capable of greater than 12 population doublings.

One embodiment of aspect two is wherein the manipulation is genetic manipulation. In a specific embodiment of aspect two the genetic manipulation is viral-induced genetic manipulation. In a most specific embodiment of aspect two the viral-induced genetic manipulation is accomplished using EBV, SV40 T antigen, adenovirus, or human papillomavirus. In another specific embodiment the genetic manipulation results in expression of telomerase. In still another specific embodiment the expression of telomerase is conditional expression.

A third aspect of the invention is a method of making an I-Amnion-derived Cellular Cytokine Solution (I-ACCS) comprising a) culturing for a first time I-AMP cells until they reach confluence; b) changing the culture medium; c) culturing for a second time the I-AMP cells; and d) collecting the culture medium of step (c) to obtain I-ACCS.

In a specific embodiment of aspect three the I-AMP cells in step (c) are cultured for about 3-6 days.

A fourth aspect of the invention is an I-ACCS solution made by the method of aspect three.

A fifth aspect of the invention is a cell bank comprising cryovials of cryopreserved cells, wherein the cryovials comprise the composition of aspect one.

A sixth aspect of the invention is a manufacturing unit comprising a cryovial of cryopreserved cells obtained from the cell bank of aspect five.

A seventh aspect of the invention is a manufacturing process comprising the step of combining the manufacturing unit of aspect six with cell culture medium.

An eighth aspect of the invention is a composition made by the manufacturing process of the seventh aspect.

A ninth aspect of the invention is a composition of aspect eight which is I-ACCS.

A tenth aspect of the invention is the composition of aspect nine which is a pharmaceutical composition.

An eleventh aspect of the invention is a kit comprising the pharmaceutical composition of aspect ten.

A twelve aspect of the invention is a therapeutic component comprising the pharmaceutical composition of aspect ten.

A thirteenth aspect of the invention is therapeutic component of aspect twelve, suitable for use in treating wounds.

### DEFINITIONS

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons). In recombinant DNA technology, genes inserted into expression vectors typical do not include the introns.

As used herein, the term “protein marker” means any protein molecule characteristic of a cell or cell population. The protein marker may be located on the plasma membrane of a cell or in some cases may be a secreted protein.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types under normal circumstances.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “Amnion-derived Multipotent Progenitor cell” or “AMP cell” means a specific population of cells selected from the amnion epithelial cells which are derived from the amnion. AMP cells have the following characteristics. They secrete the cytokines VEGF, Angiogenin, PDGF and TGFβ2 and the MMP inhibitors TIMP-1 and TIMP-2. The physiological range of the cytokines in the unique combination is as follows: ˜5-16 ng/mL for VEGF, ˜3.5-4.5 ng/mL for Angiogenin, ˜100-165 μg/mL for PDGF, ˜2.5-2.7 ng/mL for TGFβ2, ˜0.68 μg mL for TIMP-1 and ˜1.04 μg/mL for TIMP-2. In addition, AMP cells have not been cultured in the presence of any non-human animal-derived products or substances, making them and cell products derived from them suitable for human clinical use as they are not xeno-contaminated. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are selected, do not react with antibodies to the stem/progenitor cell markers c-kit (CD117) and Thy-1 (CD90). AMP cells lack c-kit (CD117) expression as well, although Thy-1 expression increases as the cells are cultured. Finally, AMP cells are not immortal. Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells. AMP cells have previously been described as “amnion-derived cells” (see U.S. Provisional Application Nos. 60/666,949, 60/699,257, 60/742,067, U.S. Provisional Application Nos. 60/813,759, U.S. application Ser. No. 11/392,892, U.S. application Ser. No. 11/724,094, and PCTUS06/011392, each of which is incorporated herein in its entirety).

As used herein, the term “population doubling” means the number of times a cell population doubles in cell number.

The term “immortalized” as used herein means a population of cells, for example AMP cells, that have been manipulated such that they are capable of a significantly greater number of population doublings than the population of cells was capable of prior to the manipulation.

The term “Immortalized Amnion-derived Multipotent Progenitor cells” or “I-AMP cells” as used herein means a population of AMP cells that has been manipulated such that it is capable of greater than 12 population doublings.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “expanded”, in reference to cell compositions, means that the cell population constitutes a significantly higher yield of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “cell bank”, “master cell bank” or “banked cells” means a culture of fully characterized cells processed and stored together to ensure uniformity and stability and which may be used to prepare the working cell banks for production.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells may be expanded in culture by being grown in the growth medium described herein. When such primary cells are subcultured, each round of subculturing is referred to as a passage. As used herein, “primary culture” means the freshly isolated cell population.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein.

As used herein, the term “Amnion-derived Cellular Cytokine Solution” or “ACCS” means conditioned medium that has been derived from AMP cells.

As used herein, the term “I-Amnion-derived Cellular Cytokine Solution” or “I-ACCS” means conditioned medium that has been derived from I-AMP cells.

The term “lysate” as used herein refers to the composition obtained when cells, for example, AMP cells or I-AMP cells, are lysed and, optionally, the cellular debris (e.g., cellular membranes) is removed. Lysis may be achieved by mechanical means, by freezing and thawing, by sonication, by use of detergents, such as EDTA, or by enzymatic digestion using, for example, hyaluronidase, dispase, proteases, and nucleases. In some instances, it may be desirable to retain the cell membranes after lysis.

The term “physiological level” as used herein means the level that a substance in a living system is found, for example, in the circulatory system or in a particular microenvironment or biological niche in the living system, and that is relevant to the proper functioning of biochemical and/or biological processes.

As used herein, the term “pooled” means a plurality of compositions that have been combined to create a new composition having qualities such as more constant or consistent characteristics as compared to the non-pooled compositions. For example, pooled AMP cells have more constant or consistent characteristics compared to non-pooled AMP cells.

As used herein, the term “substrate” means a defined coating on a surface that cells attach to, grow on, and/or migrate on. As used herein, the term “matrix” means a substance that cells grow in or on that may or may not be defined in its components. The matrix includes both biological and non-biological substances. As used herein, the term “scaffold” means a three-dimensional (3D) structure (substrate and/or matrix) that cells grow in or on. It may be composed of biological components, synthetic components or a combination of both. Further, it may be naturally constructed by cells or artificially constructed. In addition, the scaffold may contain components that have biological activity under appropriate conditions.

The term “cell product” or “cell products” as used herein refers to any and all substances made by and secreted from a cell, including but not limited to, protein factors (i.e. growth factors, differentiation factors, engraftment factors, cytokines, morphogens, proteases (i.e. to promote endogenous cell delamination, protease inhibitors), extracellular matrix components (i.e. fibronectin, etc.).

As used herein, the term “manufacturing unit” means a defined composition contained in a cryovial that comprises between about 10×10⁶/mL and 1-4 mLs/vial of I-AMPs cells, such manufacturing unit being suitable for seeding a cell culture vessel intended for the manufacture of I-ACCS.

The term “therapeutically effective amount” means that amount of a therapeutic agent necessary to achieve a desired physiological effect (i.e. promote wound healing).

As used herein, the term “therapeutic component” means a component of the composition which exerts a therapeutic benefit when the composition is administered to a subject.

As used herein, the term “pharmaceutically acceptable” means that the components, in addition to the therapeutic agent, comprising the formulation, are suitable for administration to the patient being treated in accordance with the present invention.

As used herein, the term “therapeutic protein” includes a wide range of biologically active proteins including, but not limited to, growth factors, enzymes, hormones, cytokines, inhibitors of cytokines or other proteins factors, blood clotting factors, peptide growth and differentiation factors.

The term “transplantation” as used herein refers to the administration of a composition comprising cells, including cells that may be in an undifferentiated, partially differentiated, fully differentiated, and/or immortalized form, or a combination thereof, into a human or other animal.

As used herein, the terms “a” or “an” means one or more; at least one

As used herein, the term “adjunctive” means jointly, together with, in addition to, in conjunction with, and the like.

As used herein, the term “co-administer” can include simultaneous or sequential administration of two or more agents.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, epidural, intracerebral and intrasternal injection or infusion.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein, a “wound” is any disruption, from whatever cause, of normal anatomy (internal and/or external anatomy) including but not limited to traumatic injuries such as mechanical (i.e. contusion, penetrating), thermal, chemical, electrical, concussive and incisional injuries; elective injuries such as operative surgery and resultant incisional hernias, fistulas, etc.; acute wounds, chronic wounds, infected wounds, and sterile wounds, as well as wounds associated with disease states (i.e. ulcers caused by diabetic neuropathy or ulcers of the gastrointestinal or genitourinary tract). A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones. In accordance with the subject invention, “wound healing” refers to improving, by some form of intervention, the natural cellular processes and humoral substances of tissue repair such that healing is faster, and/or the resulting healed area has less scaring and/or the wounded area possesses tissue strength that is closer to that of uninjured tissue and/or the wounded tissue attains some degree of functional recovery.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 2007, “Current Protocols in Molecular Biology” Volumes I-IV; Celis, ed., 2005, “Cell Biology: A Laboratory Handbook” Volumes I-III; Coligan, ed., 2007, “Current Protocols in Immunology”; Gait ed., 1984, “Oligonucleotide Synthesis”; Hames & Higgins eds., 1991, “Nucleic Acid Hybridization”; Hames & Higgins, eds., 1985, “Transcription And Translation: A Practical Approach”; Freshney, ed., 2006, “Animal Cell Culture” 2^{nd} Ed.; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Obtaining and Culturing of Cells

Various methods for isolating cells from the amnion of the placenta are described in the art (see, for example, US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179). Once the cells are isolated from the amnion, they are used to select and culture AMP cells (see below).

AMP cell compositions are prepared using the steps of a) recovery of the amnion from the placenta, b) dissociation of the epithelial cells from the amniotic membrane, c) culturing of the dissociated epithelial cells in a basal medium such as IMDM with the addition of a naturally derived or recombinantly produced human protein, preferably human serum albumin; d) selecting the adherent cells (the AMP cells) and discarding the non-adherent cells from the cell culture, and optionally e) further proliferation of the cells, optionally using additional additives and/or growth factors (i.e. recombinant human EGF). For details, see U.S. Pat. No. 8,088,732 and No. 8,058,066, both incorporated by reference herein.

Culturing of the AMP cells—The AMP cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, ADVANCED™ DMEM enhanced basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® [symbol] T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology. In preferred embodiments, the basal media is IMDM highly enriched basal medium and the human protein is human serum albumin at a concentration of at least 0.5% and up to 10%. In particular embodiments, the human serum albumin concentration is from about 0.5 to about 2%. The human serum albumin may come from a liquid or a dried (powder) form and includes, but is not limited to, recombinant human serum albumin, PLASBUMIN® normal human serum albumin and PLASMANATE® human blood fraction (both made by Talecris Biotherapeutics).

In a most preferred embodiment, the cells are cultured using a system that is free of non-human animal products to avoid xeno-contamination. In this embodiment, the culture medium is IMDM highly enriched basal medium culture medium, with human serum albumin (PLASBUMIN® normal human serum albumin) added up to concentrations of 10%. The invention further contemplates the use of any of the above basal media wherein animal-derived proteins are replaced with recombinant human proteins and animal-derived serum, such as BSA, is replaced with human serum albumin. In preferred embodiments, the media is serum-free in addition to being non-human animal-free.

Additional proliferation—Optionally, other proliferation factors are used. In one embodiment, epidermal growth factor (EGF), at a concentration of between 0-1 μg/mL is used. In a specific embodiment, the EGF concentration is around 10 ng/mL. Alternative growth factors which may be used include, but are not limited to, TGFα or TGFβ2 (5 ng/mL; range 0.1-100 ng/mL), activin A, cholera toxin (preferably at a level of about 0.1 μg/mL; range 0-10 μg/mL), transferrin (5 μg/mL; range 0.1-100 μg/mL), fibroblast growth factors (bFGF 40 ng/mL (range 0-200 ng/mL), aFGF, FGF-4, FGF-8; (all in range 0-200 ng/mL), bone morphogenic proteins (i.e. BMP-4) or other growth factors known to enhance cell proliferation.

Immortalization of AMP Cells to Create I-AMP Cells.

Several methods exist for immortalizing mammalian cells in culture. Viral genes, including Epstein-Barr virus (EBV), Simian virus 40 (SV40) T antigen, adenovirus E1A and E1B, and human papilloma virus (HPV) E6 and E7 can induce immortalization by a process known as viral transformation. Generally, these viral genes achieve immortalization of the cell by inactivating the tumor suppressor genes that put cells into a replicative senescent state. Occasionally, these cells may become genetically unstable (aneuploid) and lose the properties of the primary cell. It is desirable but not critical that such viral-induced immortalization does not also result in transformation of the cells into a tumor cell phenotype, provided the cells are not to be used for transplantation purposes.

U.S. Pat. No. 6,358,688 describes the immortalization of human middle ear epithelial cells using human papillomaviruses wherein a non-tumorigenic immortalized cell line was created that retained phenotypic properties of middle ear epithelial cells. Maintaining the phenotypic properties of the primary cells is a desirable feature.

Another method to immortalize cells is through expression of the telomerase reverse transcriptase protein (TERT), particularly in those cells most affected by telomere length (e.g., human cells). This protein is inactive in most somatic cells, but when hTERT is exogenously expressed, the cells are able to maintain telomere lengths sufficient to avoid replicative senescence. Analysis of several telomerase-immortalized cell lines has verified that the cells maintain a stable genotype and retain critical phenotypic markers.

Another method for immortalizing cells is conditional expression of telomerase. Several techniques are described to accomplish this (see, for example, Gray, D. C., et al., BMC Biotechnology 2007, 7:61; Szulc, J., et al., Nature Methods 2006 3(2):109-116; Geurts, A. M., et al., BMC Biotechnology 2006, 6:30; and Vallier, L., et al., PNAS 2001 98(5):2467-2472). Again, maintaining the phenotypic properties of the primary cells is a desirable feature.

US 20070264243 describes reversibly immortalized hepatocytes using a recombinant retrovirus containing an oncogene capable of inducing tumorigenic growth, flanked by recombinase target sites. Excision of the oncogene from the immortalized cells is accomplished by site-specific recombination following introduction into the cells of a gene encoding the recombinase that specifically recognizes the recombinase target sites. After site-specific recombination and oncogene excision, cell proliferation stops and the cells develop the characteristics of differentiated hepatocytes. Moreover, the cells possess minimal oncogenic potential as determined by in vitro assays.

US 20070237754 describes a method for transiently immortalizing cells in which immortalizing proteins are introduced into the cells from the exterior of the cell. The immortalizing proteins described are telomere proteins which, when expressed in the cell, ensure that the corresponding cell remains able to replicate without limit. Because the protein is being added to the cells and not being produced within the cell due to, for example, viral-induced genetic manipulation, the immortalization is transient rather than permanent.

US 20070116691 describes the conditional immortalization of long-term stem cells, in particular, hematopoietic stem cells (It-HSCs). The conditional immortalization is obtained by transfecting the cells with a nucleic acid molecule comprising an inducible proto-oncogene or a biologically active fragment or homologue thereof that is capable of promoting cell survival and proliferation, transfecting the cells with a nucleic acid molecule encoding a protein that inhibits apoptosis of the cell, and expanding the transfected cells in the presence of a combination of stem cell growth factors under conditions whereby the inducible proto-oncogene is active.

Many commercially available kits are also useful in creating the I-AMP cells of the invention. These kits are described below in the Examples section.

Generation of Conditioned Medium (I-ACCS) from I-AMP Cells

Generation of I-ACCS—The I-AMP cells of the invention can be used to generate I-ACCS. In one embodiment, the 1×10⁶/mL I-AMP cells are seeded into T75 flasks containing between 5-30 mL culture medium, preferably between 10-25 mL culture medium, and most preferably about 10 mL culture medium. The cells are cultured until confluent, the medium is changed and in one embodiment the I-ACCS is collected 1 day post-confluence. In another embodiment the medium is changed and I-ACCS is collected 2 days post-confluence. In another embodiment the medium is changed and I-ACCS is collected 4 days post-confluence. In another embodiment the medium is changed and I-ACCS is collected 5 days post-confluence. In a preferred embodiment the medium is changed and I-ACCS is collected 3 days post-confluence. In another preferred embodiment the medium is changed and I-ACCS is collected 3, 4, 5, 6 or more days post-confluence. Skilled artisans will recognize that other embodiments for collecting I-ACCS from I-AMP cell cultures, such as using other tissue culture vessels, including but not limited to cell factories, flasks, hollow fibers, bioreactors or suspension culture apparatuses, or collecting I-ACCS from sub-confluent and/or actively proliferating cultures, are also contemplated by the methods of the invention. It is also contemplated by the instant invention that the I-ACCS be cryopreserved following collection. It is also contemplated by the invention that I-ACCS be lyophilized following collection. It is also contemplated that I-ACCS be formulated in a sustained-released formulation. Skilled artisans are familiar with cryopreservation, lyophilization and sustained-release methodologies.

Generation of Cell Banks of I-AMP Cells

Master and working cell banks can be prepared using procedures familiar to skilled artisans. Briefly, a Master Cell Bank (MCB) is made using defined and characterized immortalized I-AMP cells which are cultured in medium whose supplements are human-only (derived from human, recombinant human, etc.) to expand the cell population. Generally, the cells are cultured in sufficient quantities to make 100-400 vials of frozen cells (10×10⁶/mL and 1-4 mLs/vial) from one cell batch, although skilled artisans will recognize that greater or lesser numbers of cells may be included in the cryovials. Cell batches are created by pooling the cells from one batch of cultured cells, mixing well and then pipetting into the vials. The goal is to make every vial of frozen cells in the bank the same. The vialed cells are then frozen (preferably using a controlled-rate freezer) and stored in liquid nitrogen. The Working Cell Bank (WCB) is made the same way as the MCB except that it is started with one vial of the MCB. Vials obtained from the WCB are use to seed cell culture vessels (i.e. cell factories or bioreactors) for the manufacture of I-ACCS.

Pharmaceutical Compositions

The present invention provides pharmaceutical compositions of I-ACCS, optionally in a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Formulation, Dosage and Administration

Compositions comprising I-ACCS may be administered to a subject to provide and/or induce various cellular or tissue functions, for example, to treat wounds due to burn, trauma, surgery, etc. As used herein “subject” may mean either a human or non-human animal.

Such compositions may be formulated in any conventional manner optionally using one or more physiologically acceptable carriers further optionally comprising excipients and auxiliaries. Proper formulation is dependent upon the route of administration chosen. The compositions may be packaged with written instructions for their use in treating, for example, wounds. The compositions may also be administered to the recipient in one or more physiologically acceptable carriers. Carriers for I-ACCS may include carriers suitable for sustained-release of I-ACCS. Carriers may also include those suitable for lyophilization.

Treatment Kits

The invention also provides for an article of manufacture comprising packaging material and a pharmaceutical composition of the invention contained within the packaging material, wherein the pharmaceutical composition comprises compositions of I-ACCS. The packaging material comprises a label or package insert which indicates that the I-ACCS can be used for treating, for example, wounds.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees centigrade, and pressure is at or near atmospheric.

### Example 1

### Preparation of AMP Cell Compositions

Recovery of AMP cells—Amnion epithelial cells were dissociated from starting amniotic membrane using the dissociation agent PXXIII. The average weight range of an amnion was 18-27 g. The number of cells recovered per g of amnion was about 10-15×10⁶.

Method of selecting AMP cells: Amnion epithelial cells were plated immediately upon isolation from the amnion. After ˜2-3 days in culture, non-adherent cells were removed and the adherent cells were kept. The adherent cells represent about 30% of the plated cells. This attachment to a plastic tissue culture vessel is the selection method used to obtain the desired population of AMP cells. Selected AMP cells were cultured until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2

### Immortalization of AMP Cells Using Epstein-Barr Virus (EBV)

The I-AMP cells of the invention are created by immortalizing AMP cells using Epstein Barr virus immortalization techniques familiar to skilled artisans. For example, AMP cells are immortalized with Epstein Barr Virus as described, for example, in Current Protocols in Molecular Biology, Unit 28.2, Isolation and Immortalization of Lymphocytes, by Paul D. Ling and Helen M. Hula, Publisher John Wiley & Sons, Inc. 2005 or Pelloquin, F., et al., In Vitro Cell & Dev Biol 22(12):689-94, 1986.

### Example 3

### Immortalization of AMP Cells Using Simian virus 40 (SV40) T Antigen

The I-AMP cells of the invention are created by immortalizing AMP cells using SV40 T antigen immortalization techniques familiar to skilled artisans. For example, the AMP cells are immortalized with SV40 T antigen as describe in Lei, K-J., et al, Molecular Endocrinology 6:703-712, 1992. In addition, AMP cells are immortalized using commercially available kits such as those sold by Applied Biological Materials Inc., Applied Biological Materials Inc., 9117 Shaughnessy St, Vancouver, BC, Canada V6P 6R9 (pPromoter-SV40, cat. # G209; Adeno-SV40, cat. # G210; pRetro-E2/SV40, cat. # G211; Retro/SV40 virus, cat. #G212; pLenti/SV40, cat. # G204; Lenti/SV40 virus, cat. # G203).

### Example 4

### Immortalization of AMP Cells Using Gene-Specific Immortalization Techniques

The I-AMP cells of the invention are created by immortalizing AMP cells using gene-specific immortalization techniques. For example, AMP cells are immortalized using commercially available kits such as those sold by Applied Biological Materials Inc., Applied Biological Materials Inc., 9117 Shaughnessy St, Vancouver, BC, Canada V6P 6R9 (Lenti-Myc T58A, cat. # G216; Lenti-Myc T58A virus, cat. # G217; Lenti-p53 siRNA, cat. # G218; Lenti-p53 siRNA virus, cat. # G219; Lenti-Ras V12, cat. # G220; Lenti-Ras V12 virus, cat. # G221; Lenti-Rb siRNA, cat. # G222; Lenti-Rb siRNA virus, cat. # G223).

### Example 5

### Immortalization of AMP Cells Using Adenovirus E1A and E1

The I-AMP cells of the invention are created by immortalizing AMP cells using adenovirus immortalization techniques familiar to skilled artisans (see, for example, Hurwitz, D. R. and Chinnadurai, G., J of Virology 54(2):358-363, 1985; Douglas, J. L. and Quinlan, M. P., J of Virology 69(12):8061-65).

### Example 6

### Immortalization of AMP Cells Using Human Papillomavirus (HPV) E6 and E7

The I-AMP cells of the invention are created by immortalizing AMP cells using human papilloma virus immortalization techniques familiar to skilled artisans. For example, the AMP cells are immortalized with human papilloma virus gene E6E7 as describe in Feng, J. Y., et al., (Cancer Genet Cytogenet, 163(1):30-7, 2005.

### Example 7

### Immortalization of AMP Cells Through Expression of the Telomerase Reverse Transcriptase Protein (TERT)

The I-AMP cells of the invention are created by immortalizing AMP cells using TERT gene immortalization techniques familiar to skilled artisans. For example, the AMP cells are immortalized by expression of the telomerase reverse transcriptase gene (TERT) using, for example, the technique described in Wang, Y-L., et al (Molecular Human Reproduction, 12(7):451-60, 2006 or Zhang, X., et al, (Biochem Biophys Res Commun, 351(4):853-9, 2006. In addition, AMP cells are immortalized using commercially available kits such as those sold by Applied Biological Materials Inc., Applied Biological Materials Inc., 9117 Shaughnessy St, Vancouver, BC, Canada V6P 6R9 (Adeno-hTERT, cat. #G205; Adeno-hTERT Antisense, cat. # G208; pRetro-E1/hTERT, cat. # G206; pRetro-E1/hTERT virus, cat. # G207; pLenti-hTERT vector, cat. # G214; Lenti-hTERT virus, cat. # G200; pLenti-hTERT Antisense vector, cat. # G215; pLenti-hTERT Antisense virus, cat. # G201).

### Example 8

### Immortalization of AMP Cells Using Conditional Expression of Telomerase

AMP cells may be conditionally immortalized by the conditional expression of telomerase. Skilled artisans are familiar with such techniques. For example, lentiviral vectors containing the drug-controllable expression of polymerase (Pol) II promoter-driven expression of transgenes (i.e. telomerase) or Pol III promoter-controlled sequences encoding small inhibitory hairpin RNAs (shRNAs) are suitable methodologies for creating immortalized AMP cells. Details of this system can be found in Szulc, J., et al., Nature Methods 2006 3(2):109-116.

Another suitable methodology for creating conditionally immortalized AMP cells is the pHUSH vector system. This inducible expression vector system is used for regulated expression of shRNA, miRNA or cDNA cassettes on a single viral vector. Details of this system can be found in Gray, D. C., et al., BMC Biotechnology 2007, 7:61.

Another useful approach for creating conditionally immortalized AMP cells by conditional expression telomerase is the transposon-based gene trap system. This system incorporates the doxycycline-repressive Tet-Off (tTA) system that is capable of activating the expression of a gene (for example telomerase) which is under control of a Tet response element (TRE) promoter. Details of this system can be found in Geurts, A. M., et al., BMC Biotechnology 2006, 6:30.

Also useful for creating conditionally immortalized AMP cells is conditional gene expression using tamoxifen-dependent Cre recombinase-loxP site-mediated recombination and bicistronic gene-trap expression vectors that allow for transgene (i.e. telomerase) expression from endogenous promoters. Details of this system can be found in Vallier, L., et al., PNAS 2001 98(5):2467-2472.

### Example 9

### Immortalization of AMP Cells Using Ionizing Radiation

The I-AMP cells of the invention are created by immortalizing AMP cells using ionizing radiation immortalization techniques familiar to skilled artisans. For example, the AMP cells are immortalized with ionization radiation using the techniques described in Trott, D. A., et al., Carcinogenesis 16(2):193-204, 1995.

### Example 10

### Immortalization of AMP Cells Using Agents Capable of Altering Cell Cycle Regulation

The I-AMP cells of the invention may be created by altering various steps in the cell cycle to create a cell cycle that resembles the cell cycle exhibited by ES cells (see, for example, He, S., et al (Annu Rev Cell Div Biol 2009, 25:377-406) and Orford, K. and Scadden, D. (Nature Rev, 2008, 9:115-128) for details on the ES cell cycle). For example, the mechanisms controlling the transition from G1 to S phase are suitable targets for alteration, with a shorter G1 and faster transition into S and an increased rate of cell division being desirable. For example, this may be accomplished by inhibiting the Rb genes (Rb, p107, 130) by knockout, phosphorylation or addition of an exogenous inhibitor, and/or over expression of Cyclin D-CDK4/6.

### Example 11

### Generation of I-AMP Cell Banks

I-AMP cell Master Cell Banks (MCBs) and Working Cell Banks (WCBs) are created to provide a consistent source of cells. The I-AMP cells are expanded to the requisite density (typically ˜10×10⁷ cells/mL, ˜4 mL/vial) to fill cryovials for the cell bank. I-AMP cell banks are tested for recovery viability after cryopreservation. The cell banks are used in the manufacture of I-ACCS.

### Example 12

### Generation of I-ACCS

The I-AMP cells of the invention are used to generate I-ACCS. The I-AMP cells are prepared as described above. I-AMP cells are seeded at ˜1×10⁶ cells/mL culture medium into T75 flasks. The cells are cultured until confluent, the medium is changed and in one embodiment the ACCS is collected 1 day post-confluence. In another embodiment the medium is changed and I-ACCS is collected 2 days post-confluence. In another embodiment the medium is changed and I-ACCS is collected 4 days post-confluence. In another embodiment the medium is changed and I-ACCS is collected 5 days post-confluence. In a preferred embodiment the medium is changed and I-ACCS is collected 3 days post-confluence. In another preferred embodiment the medium is changed and I-ACCS is collected 3, 4, 5, 6 or more days post-confluence. Skilled artisans will recognize that other embodiments for collecting I-ACCS from I-AMP cell cultures, such as using other tissue culture vessels, including but not limited to cell factories, flasks, hollow fibers, bioreactors or suspension culture apparatuses for large scale manufacturing, or collecting I-ACCS from sub-confluent and/or actively proliferating cultures, are also contemplated by the methods of the invention. In addition, I-ACCS may be cryopreserved following collection. I-ACCS may also be lyophilized following collection. The I-ACCS may also be formulated in a sustained-release formulation. Skilled artisans are familiar with cryopreservation, lyophilization and sustained-release methodologies.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is:

1. A composition comprising a population of Immortalized Amnion-derived Multipotent Progenitor (I-AMP) cells, wherein the I-AMP cells are capable of greater than 12 population doublings.

2. A method of making I-AMP cells comprising:
a) isolating amnion epithelial cells from the amnion of a placenta;
b) selecting AMP cells from the amnion epithelial cells; and
c) manipulating the AMP cells to immortalize them, wherein immortalization is characterized by the I-AMP cells being capable of greater than 12 population doublings.

3. The method of claim 2 wherein the manipulation is genetic manipulation.

4. The method of claim 3 wherein the genetic manipulation is viral-induced genetic manipulation.

5. The method of 4 wherein the viral-induced genetic manipulation is accomplished using EBV, SV40 T antigen, adenovirus, or human papillomavirus.

6. The method of claim 5 wherein the genetic manipulation results in expression of telomerase.

7. The method of claim 6 wherein the expression of telomerase is conditional expression.

8. A method of making an I-Amnion-derived Cellular Cytokine Solution (I-ACCS) comprising
a) culturing for a first time I-AMP cells until they reach confluence;
b) changing the culture medium;
c) culturing for a second time the I-AMP cells; and
d) collecting the culture medium of step (c) to obtain I-ACCS.

9. The method of claim 8 wherein the I-AMP cells in step (c) are cultured for about 3-6 days.

10. An I-ACCS solution made by the method of claim 9.

11. A cell bank comprising cryovials of cryopreserved cells, wherein the cryovials comprise the composition of claim 1.

12. A manufacturing unit comprising a cryovial of cryopreserved cells obtained from the cell bank of claim 11.

13. A manufacturing process comprising the step of combining the manufacturing unit of claim 12 with cell culture medium.

14. A composition made by the manufacturing process of claim 13.

15. The composition of claim 14 which is I-ACCS.

16. The composition of claim 15 which is a pharmaceutical composition.

17. A kit comprising the pharmaceutical composition of claim 16.

18. A therapeutic component comprising the pharmaceutical composition of claim 16.

19. The therapeutic component of claim 18, suitable for use in treating wounds.
